(19) United States
(12) Patent Application Publication
(10) Pub. No.: US 2004/0115177 A1
(43) Pub. Date: Jun. 17, 2004

(54) Probiotic compositions and methods against bacterial infection in livestock animals

(76) Inventors: Delbert L. Harris, Ames, IA (US); Christian L Baum, Ames, IA (US); Alan Loynachan, Ames, IA (US)

Correspondence Address: James W Brady Jr, Dickstein Shapiro Morin & Oshinsky, 2101 L Street NW, Washington, DC, 20037-1526, US

(21) Appl. No.: 10/433,684

(22) PCT Filed: Oct. 12, 2001
(86) PCT No.: PCT/US01/31789

(51) Int. Cl. A61K 45/00; A23K 1/165; A23K 1/17

(52) U.S. Cl. 424/93.45; 424/442

(57) ABSTRACT

Methods of administering probiotic bacteria to livestock animals in an amount effective to reduce the amount of hazardous bacteria. Probiotic bacteria in an acceptable carrier are administered to the livestock at a selected time before transportation of the animal for harvest or other purposes. The invention also provides compositions comprising probiotic bacteria in an amount effective to reduce the amount of Salmonella in livestock animals.

This patent application claims the benefit of U.S. provisional patent application serial No. 60/247,020, filed Nov. 13, 2000, entitled PROBIOTIC TREATMENT OF BACTERIAL INFECTION IN LIVESTOCK ANIMALS. U.S. provisional patent application serial No. 60/247,020 is hereby incorporated by reference.

### STATEMENT REGARDING FEDERALLY SPONSORED RESEARCH OR DEVELOPMENT

The United States Government may have certain rights to this invention. This invention was made with government support under a grant awarded by the U.S.D.A., National Pork Producers Council.

### BACKGROUND OF THE INVENTION

Each year food borne Salmonella and other bacteria causes illness in between 2 and 4 million people in the United States alone. Strains of Salmonella have become more resistant to antibiotics because of their continued and dominant use in treating the bacteria in livestock animals.

Subtherapeutic levels of antimicrobial feed additives have been used in swine diets since the 1950s for improved growth rate and feed efficiency and to maintain pig performance in the presence of sub-clinical disease. The consistent effectiveness of antimicrobial feed additives has led to extensive use in the swine feeding industry. In 1991, it was estimated that at least 85% of all starter pig feeds, 75% of all grower pig feeds, 55% of all finisher pig feeds, and 20% of all sow feeds were fortified with antimicrobial feed additives. At least 17 antimicrobial feed additive products are currently approved by the U.S. Food and Drug Administration (“FDA”).

Despite their effectiveness, use of antimicrobial feed additives faces an uncertain future. There is increasing pressure from regulatory agencies and from consumer groups to curb the use of antimicrobial additives in livestock and poultry feeds. Of particular concern is that regular use of antimicrobial feed additives may lead to the development of resistant microbes that may compromise the effectiveness of antibiotics in treating both animal and human disease. European authorities issued a ban effective Jul. 1, 1999, on four prominent feed grade antimicrobials used within the European Union member countries. These additives included virginiamycin, spiramycin, tylosin and zinc bacitracin. Three of these products are currently approved and used in U.S. swine feeds. It remains an open question if these countries will apply the same ban to pork products imported from outside of Europe. In the U.S., the FDA has proposed new regulations that will restrict approval of new antimicrobial additives while the resistant bacteria issue is studied further.

Antibiotic alternatives are important to the meat producing industry's future, including the pork industry. Several human health organizations are moving toward regulations that reduce the use of antimicrobial products in animal agriculture. Safe, effective alternatives are needed to reduce the industry's reliance on antibiotics.

One alternative is “probiotic bacteria,” which encourage the growth of non-harmful bacteria by excluding the growth of pathogenic bacterial strains and preventing the spread of disease-causing bacteria in animals.

### BRIEF SUMMARY OF THE INVENTION

This invention provides methods of reducing the amount of Salmonella in a livestock animal. Also provided herein are compositions and kits comprising an effective amount of probiotic bacteria in an acceptable carrier to reduce the amount of Salmonella in a livestock animal.

In one embodiment, probiotic bacteria in an acceptable carrier are administered to a livestock animal, at least three weeks prior to shipment or transport of the animal for harvest or other purposes, in an amount effective to reduce the levels of Salmonella in the animal. In another embodiment, lactic acid bacteria are administered from at least about three weeks to about three hours prior to transport of the animal. In another embodiment, a probiotic cocktail containing lactic acid producing bacteria, acetic acid producing bacteria, and mixed acid producing bacteria in an acceptable carrier is administered to a livestock animal in an amount sufficient to reduce the amount of Salmonella in the animal. Compositions and kits comprising an effective amount of probiotic bacteria in an acceptable carrier to reduce the amount of Salmonella in a livestock animal are also provided.

These and other features of the invention will be more readily understood from the following detailed description, which is provided in connection with the accompanying figures.

### BRIEF DESCRIPTION OF THE DRAWINGS

FIG. 1 is a chart depicting the anti-Salmonella antibody level in swine subject to treatment with Lactobacillus paracasei paracasei GS-1, Lactobacillus paracasei paracasei GS-2, and uninnoculated milk.

FIG. 2 is a chart depicting the Salmonella serum IgA levels in swine subject to treatment with Lactobacillus paracasei paracasei GS-1 compared to swine treated with uninnoculated milk.

FIG. 3 is a western immunoblot of serum samples from pigs treated with probiotic bacteria to test for the presence of serum antibodies toward Lactobacillus paracasei paracasei GS-1 and Lactobacillus paracasei paracasei GS-2.

FIG. 4 depicts the results of an in vitro spot test conducted to evaluate bactericidal effects of treatment in accordance with the invention.

### DETAILED DESCRIPTION OF THE INVENTION

Reduction of antibiotic use in livestock animals is of increasing concern in the food production industry. The use of probiotic therapies for livestock reduces the presence of unwanted and dangerous bacterial infections, such as by hazardous bacteria like Salmonella and E. coli. Beneficial bacteria are used as “probiotics,” non-pathogenic bacteria that compete against and replace pathogenic bacteria when administered to livestock animals. Probiotic bacteria reduce the levels of pathogenic bacteria in livestock animals thereby reducing the risk of infection to humans ingesting livestock animal meat products. The use of probiotic bacteria reduces the need for antibiotics and therefore reduces the level of antibiotic resistant bacteria in the livestock animal.

Colonization of probiotic organisms in livestock animals may occur by multiple routes including passing directly through the gastrointestinal system from oral uptake, or by phagocyte associated transfer in the proximal GI tract (i.e., oral cavity, pharynx, and tonsils) followed by transport to and release in the gut. Livestock animals are especially susceptible to extraintestinal dissemination of Salmonella, and other pathogenic organisms, during holding and transport.

Current techniques for transporting animals require co-mingling of animals in conditions that can result in rapid dissemination of pathogenic bacteria from infected to healthy animals. Transport of livestock animals also can alter the permeability of the gut resulting in greater translocation of bacteria and antigens, and alteration of the microbial population in the gut (i.e., production of less lactic and acetic acid producing bacteria and more coliform bacteria). These conditions increase the likelihood of cross-contamination by Salmonella and other pathogenic bacteria. Thus, livestock subjected to the trauma and stress of transport and holding prior to slaughter are even more susceptible to contamination by pathogenic bacteria.

In accordance with an embodiment of the invention, a composition containing Lactobacillus paracasei paracasei in an acceptable carrier is administered to swine at least about three weeks prior to shipment of the animal in an amount effective to reduce the amount of Salmonella in the animal both before and after harvest. Examples of Lactobacillus paracasei paracasei include Lactobacillus paracasei paracasei GS-1 (ATCC Number______) and Lactobacillus paracasei paracasei GS-2 (ATCC Number______), both available from the American Type Culture Collection, Manassas, Va. However, any strain of Lactobacillus paracasei paracasei is suitable for use in the present invention. In another embodiment of the invention, the Lactobacillus paracasei paracasei is administered daily beginning at least three weeks prior to transport of the animal for harvest in an amount effective to reduce the amount of Salmonella bacteria in the swine both before and after harvest. The probiotic bacteria may be delivered in an acceptable carrier via a food route of administration (e.g., milk product, water, feed, or any suitable bacteriologic medium useful for propagating probiotic bacteria) or by a medicinal route of administration (e.g., oral or intranasal innoculation). Acceptable carriers for the probiotic bacteria include feed products for the livestock animal, including, for example, milk or yogurt cultures. In another embodiment of this invention, the concentration of probiotic bacteria in the carrier is from about 10² colony forming units (“CFU”) per ml to about 10¹⁰ cfu/ml for Lactobacillus paracasei paracasei. Alternatively, the concentration of probiotic bacteria in the carrier is from about 10² cfu/ml to about 10⁷ cfu/ml. The total volume of the probiotic bacteria and carrier administered to the livestock animal can be, for example, from at least about 1 to about 5000 mls per day.

A dry probiotic culture can also be produced and added to feed by the process of lyophilization. Lyophillized probiotic bacteria may be delivered to animals by any suitable route of administration including via dry feed and water. By administering probiotic therapy in advance of transport, significant levels of hazardous bacteria such as Salmonella, are reduced in the livestock pre and post slaughter.

Lactobacillus paracasei paracasei for use in the present invention can be isolated by a variety of microbiological techniques. For example, Lactobacillus paracasei paracasei can be isolated by scraping the mucosal lining of the gastrointestinal tract of an antibiotic free conventional pig followed by selection on MRS agar incubated in an anaerobic chamber at 37° C. for 24 hours. Lactobacillus paracasei paracasei cultures can be prepared by any suitable technique for use with an acceptable carrier. The following is an exemplary method that can be used to prepare a probiotic bacterial culture in a yogurt carrier. High passage isolates of probiotic bacteria are obtained by, for example, sub-culturing a stock culture in growth media (e.g., MRS broth). Yogurt starter cultures (e.g., 700 ml of milk (2% fat), supplemented with 2% (w/v) dextrose) are prepared, sterilized, and inoculated with the probiotic stock culture. The daily titer of the probiotic bacteria in each yogurt culture can be determined by well-known techniques (i.e., serial dilutions of the culture on agar plates) to adjust the concentration of probiotic bacteria to the desired level. The above method of preparing probiotic yogurt cultures can be varied depending on the particular bacterial growth conditions and requirements. Yogurt cultures can be administered orally to livestock animals once a day in addition to a conventional diet. The probiotic bacteria are combined with the carrier by, for example, mechanical agitation.

Administration of probiotic bacteria to livestock animals significantly reduces the level of pathogenic bacteria, such as Salmonella, in the animals as shown in Table 1. Swine were intranasaly innoculated with a challenge dose of 10⁸-10⁹ Salmonella typhimurium organisms per ml followed by administration of Lactobacillus paracasei paracasei GS-1 in a yogurt carrier on the indicated day (Table 1). The CFU of Salmonella in the indicated tissue was measured before (Control CFU) and after (Probiotic CFU) administration of the probiotic Lactobacillus paracasei paracasei GS-1 to the animals. As shown in Table 1 (Control CFU/Probiotic CFU), administration of lactic acid producing bacteria reduces levels of Salmonella by about 5 to 10 fold in the mesenteric lymph nodes (MNL)and by out 1,000 to about 10,000 fold in the cecum over a period of two to nine days after administration of lactic acid producing bacteria:

TABLE 1
Reduction in S. typhimurium in pigs
following innoculation with Lactobacillus

| Days after inoculation | | Control CFU/Probiotic | |
|---|---|---|---|
| w/ S. typhimurium | Tissue | CFU | |
| 2 | Cecum contents | 1000 | fold |
| 9 | MLN | 10 | fold |
| 9 | Cecum contents | 10,000 | fold |
| 27 | MLN | — | |
| 27 | Cecum contents | 20 | fold |

In another embodiment of the invention, probiotic bacteria, such as lactic acid, acetic acid, and mixed organic acid producing bacteria, are provided to a livestock animal in an acceptable carrier in an amount sufficient to reduce the level of pathogenic bacteria in the animal. The lactic acid, acetic acid, and mixed organic acid producing bacteria may be combined to form a “probiotic cocktail.” The probiotic cocktail may include any combination of lactic acid, acetic acid, or mixed organic acid producing bacterial species. Lactic acid producing bacteria include, for example, Lactobacillus, Lactococcus, Bifidobacteria, or Proprionbacteria among others. Acetic acid producing bacteria include Streptococcus, Peptostreptococcus, and Eubacterium. Mixed acid producing bacteria include Bacteriodes.

In another embodiment of the invention, the lactic acid producing bacteria are Lactobacillus paracasei paracasei, the acetic acid producing bacteria are Streptococcus alacyolyticus, and the mixed acid producing bacteria are Bacteroides ovatus. Example of Streptococcus alacyolyticus include, ATCC Number______available from the American Type Culture Collection, Manassas, Va. Examples of Bacteroides ovatus include ATCC Number______available from the American Type Culture Collection, Manassas, Va. Streptococcus alacyolyticus and Bacteroides ovatus may also be isolated from livestock animals by a variety of methods. The following exemplary method can be used to isolate Bacteroides ovatus. Euthenized pigs are aseptically opened and 8 inches of the colon (near the apex) is ligated. A 1 g sample of the colon contents is placed into 99 ml of anaerobic dilution medium under deoxygenated carbon dioxide. The sample is blended for 2 minutes under deoxygenated carbon dioxide and anaerobically diluted to get viable counts of the bacteria. Dilutions are plated onto MRS, pre-reduced blood agar plates, and blood agar plates followed by inoculation into anaerobic roll tubes. The media is incubated both anaeobically (i.e., the MRS, pre-reduced blood agar plates, and roll tubes) and aerobically (i.e., the blood agar plates) at 37° C. until bacterial colonies exhibit substantial growth. Isolates are picked from the formed colonies and identified. The following exemplary method can be used to isolate Streptococcus alacyolyticus. Pig colon samples are obtained as described above. The colon is dissected with scissors to make a flat piece of tissue. The tissue is washed with PBS followed by removal of colonic disks. The colonic disks are placed into 99 ml of anaerobic dilution medium under deoxygenated carbon dioxide. The sample is corked and violently rocked back and forth 10 times and transferred to a fresh 99 ml bottle of anaerobic dilution fluid. Samples are blended, diluted and plated as described above with reference to Bacteroides ovatus. Bacteroides ovatus and Streptococcus alacyolyticus can be identified by standard techniques (e.g., gas chromatography, metabolic profile).

The concentration and amount of each probiotic bacteria in the probiotic cocktail may be the same as described above with regard to Lactobacillus paracasei paracasei. The probiotic bacteria of the probiotic cocktail are combined with the carrier (e.g., milk product, water, or feed) by simple mixing.

In another embodiment of the invention, the probiotic cocktail is administered to livestock animals from at least about three weeks to about three hours prior to transport for harvest or other purposes. In another embodiment of the invention, the probiotic cocktail is administered daily beginning at least about three weeks prior to transport. In yet another embodiment, the probiotic cocktail is administered to the livestock just prior to transport where intermingling of healthy and infected animals may occur.

A further embodiment of the invention provides kits for reducing the amount of Salmonella in a livestock animal. The kits comprise at least one container, containing a probiotic cocktail consisting essentially of lactic acid producing bacteria, acetic acid producing bacteria, and Bacteroides in an amount effective to reduce the amount of Salmonella in a livestock animal before and after harvest. In another embodiment of the invention, the probiotic bacteria are present in an amount of at least about 10⁴ CFU of Lactobacillus paracasei paracasei, at least about 10⁴ CFU of Streptococcus alactolyticus, and at least about 10⁴ CFU of Bacteroides ovatus. Alternatively, the kits further comprise an acceptable carrier (e.g., milk product, water, or feed).

In yet another embodiment of the invention, a method of transporting a livestock animal is provided wherein a composition containing a probiotic cocktail in an acceptable carrier is administered to the animal prior to transport of the animal. In another embodiment of the invention, the probiotic bacteria are Lactobacillus paracasei paracasei. In another embodiment of the invention, the probiotic bacteria are Lactobacillus paracasei paracasei, Streptococcus alactolyticus, and Bacteroides ovatus. Probiotic therapy is beneficial to all forms of livestock (e.g., cattle, chickens, swine, sheep, goats), but will be described below in reference to swine for exemplary purposes.

It is to be understood that the application of the teachings of the present invention to a specific problem or environment will be within the capabilities of one having ordinary skill in the art in light of the teachings contained herein. Examples of the products of the present invention and processes for their use appear in the following examples.

### EXAMPLE 1

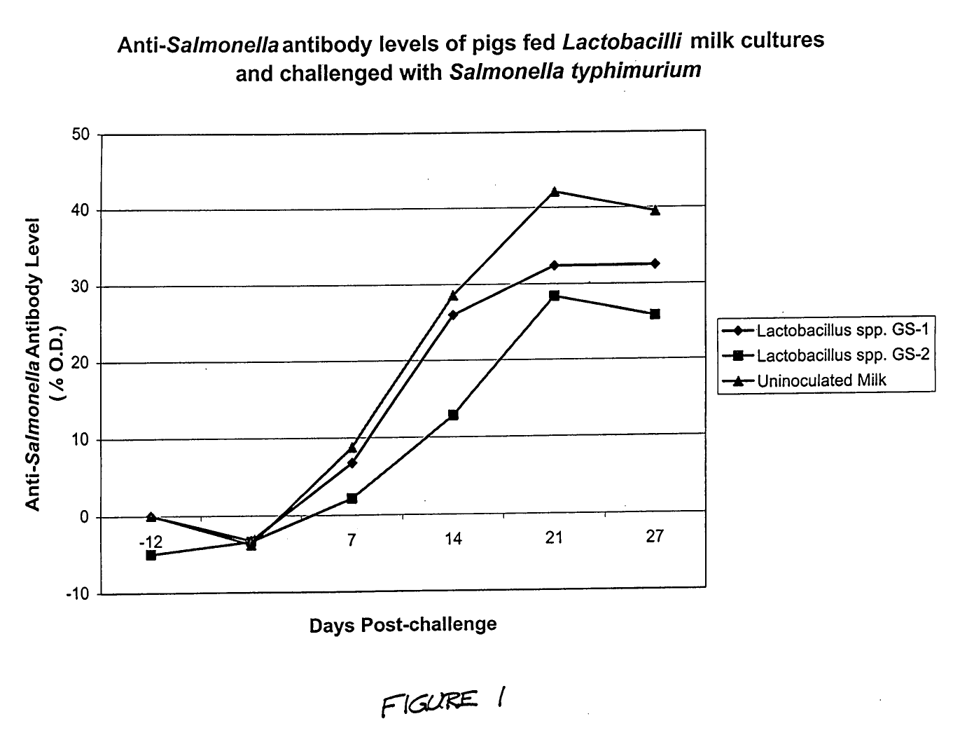

Serum antibody levels towards Salmonella were evaluated in pigs fed milk cultures of Lactobacillus spp. GS-1 (n=10), Lactobacillus spp. GS-2 (n=5) or uninoculated milk (n=10). Four-week-old pigs were fed 500 to 1000 mls of probiotic milk cultures daily for 8 days. On the 8^{th} day of milk feeding, animals were challenged intranasally with 10⁸ colony forming units (cfu) of S. typhimurium. Pigs were fed respective milk cultures for 27 days following inoculation with S. typhimurium. Blood was collected 4 days prior to milk feeding and on days −1, 7, 14, 21 and 27 relative to S. typhimurium challenge. Serum from all blood samples was tested for antibodies toward Salmonella using the Mix-ELISA assay. As shown in FIG. 1, after inoculation with S. typhimurium, pigs fed Lactobacillus spp. GS-1 and Lactobacillus spp. GS-2 had significantly lower % O.D. on average than animals fed uninoculated milk.

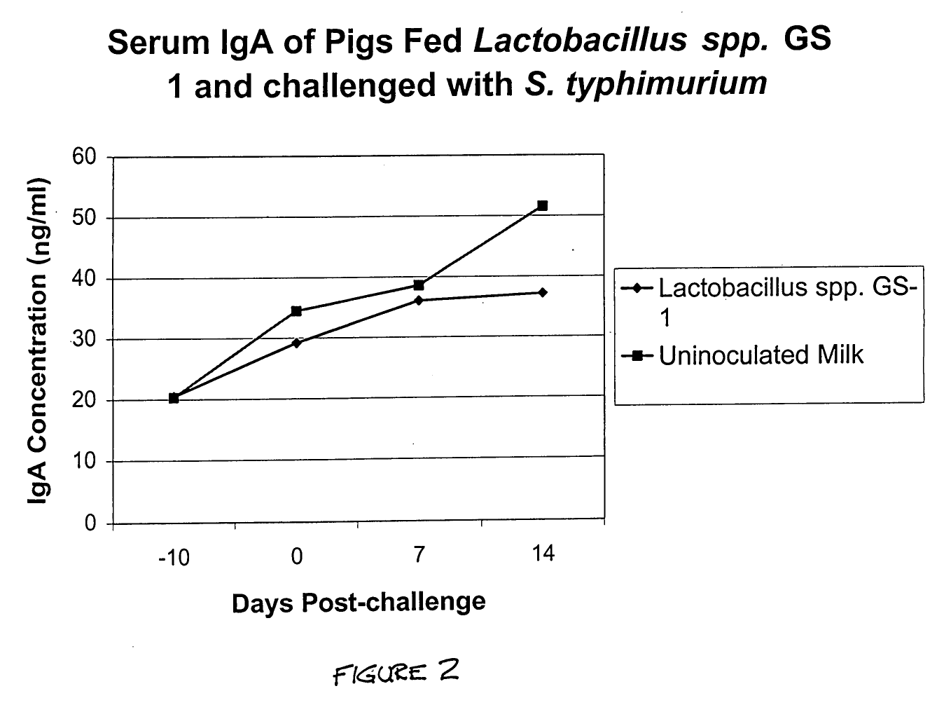

Referring to FIG. 2, serum IgA levels were evaluated in pigs fed milk cultures of Lactobacillus spp. GS-1 (n=10) and uninoculated milk (n=10) as described above. Samples collected on days −10, −1, 7 and 14 relative to inoculation with S. typhimurium were tested using a commercial kit for IgA concentration (Bethyl Laboratories, Montgomery, Tex.). As shown in FIG. 2, after inoculation with S. typhimurium, pigs fed Lactobacillus spp. GS-1 had lower concentrations of S. typhimurium serum IgA compared to animals fed uninoculated milk.

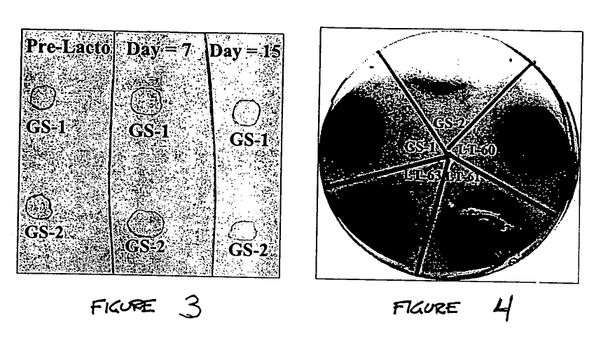

A western immunoblot was performed to evaluate the presence of serum antibodies toward Lactobacillus spp. GS-1 and Lactobacillus spp. GS-2 (FIG. 3). Pigs that were fed Lactobacilli milk cultures as described above were not challenged with S. typhimurium. Pooled serum samples (n=5) were collected on days −4, 7, and 15 relative to milk culture feeding. No serum antibodies were detected to either strain of Lactobacillus as shown in FIG. 4. Thus, the probiotic Lactobacillus bacteria did not contaminate the animals fed the Lactobacillus milk cultures.

### EXAMPLE 2

Trial 1: Fifteen 4-week-old pigs that were culture-negative for Salmonella were identified with a numbered ear tag and randomly assigned to each of three treatment groups (A, B, and C). Treatment groups A and C were repeated in Trial 2. Group A was fed daily 24-hour milk culture of Lactobacillus spp. strain GS-1. Group B was fed a 24-hour milk culture of Lactobacillus spp. strain GS-2. Group C was fed uninoculated milk. Rectal swabs were collected on days 31 12, 2, 4, 7, 9, 11, 14, 17, 19, 21, and 24 relative to challenge. Five 5-gram samples of feces were collected on the same days as rectal swabs and on day −1 relative to challenge. Palatine tonsil swabs were collected on days 15 and 21 relative to challenge. All groups were challenged intranasally with 3×10⁸ colony forming units (CFU) of Salmonella typhimurium (strain B) on day 8. Pooled pen fecal, rectal swabs and tonsil swabs were cultured for the presence of Salmonella.

Samples were pre-enriched in buffered peptone water (BPW) at 37° C. for 18-24 hours. After pre-enrichment, 100 μl was transferred to 9.9 ml Rappaport-Vassiliadis (RV) broth and incubated at 42° C. for 18-24 hr. After incubation in RV broth, a sterile cotton swab was used to transfer an aliquot of the cultures onto XLD agar plates. The agar plates were streaked for isolation and incubated at 37° C. for 24 hours. Colonies suspected to be Salmonella spp. were picked and transferred to Kliglers's iron agar, SIM semi-solid agar, phenylalanine agar, lysine iron agar and trypticase soy agar slants and incubated for 37° C. for 24 hours. Presumptive Salmonella colonies were tested for “O” antigens by agglutination with typing antisera. Identification of Salmonella from serogroup B were considered to be the challenge strain.

Trial 2: Twenty-five 4-week-old pigs that were culture-negative for Salmonella were identified. Ten animals were randomly assigned to two treatment groups: Group A and Group C as in Trial 1. Five animals were randomly assigned to a fourth group, Group D (unchallenged control). Animals in the Group A and Group C administered Lactobacillus spp. strain GS-1 as in Trial 1. Animals in Group D were administered Lactobacillus spp. strain GS-1 as in Group A. Rectal swabs were collected on days −1, 2, 4, 7,9 11, 14, 17, 19, 21, 23, 25, and 27 relative to challenge. Pen fecal samples were collected on the same days as rectal swabs except on day 23 when no pen fecal was collected. Tonsil swabs were collected on days −1, 2, 5, 7, 9, 11, 15, 21, 23, and 27 relative to challenge. Animals in the Group A and Group B were challenged as in Trial 1. Pooled pen fecal samples, rectal swabs, and tonsil swabs were cultured for the presence of Salmonella as in Trial 1. Three (3) and two (2) animals from Group A and Group C were randomly selected and euthanized on day 2 and day 9, respectively. The trial was terminated 27 days after challenge.

As shown in Tables 2-4 below, groups A and B demonstrated significantly reduced duration and number of animals culture-positive for Salmonella in the feces and tonsil compared to Group C. Salmonella was not recovered from any fecal or tonsil samples cultured from Group D. In Trials 1 and 2, 15 animals received Lactobacillus spp. strain GS-1 and were challenged. Of the 10 animals that remained until the study was terminated, Salmonella was never recovered from 5 animals on any day. Likewise, in Group B, there was one individual animal from which Salmonella was not recovered on any day. In Group C, Salmonella was recovered from all of the animals on at least one day after challenge.

TABLE 2
Salmonella isolation from pig feces

| | Day (Challenge = 0) | | | | | | | |
|---|---|---|---|---|---|---|---|---|
| Group | 2 | 4 | 7 | 9 | 11 | 14 | 21 | 24 |
| A | 5/12^{a} | 1/12^{a} | 1/12 | 2/10^{a} | 1/10^{a} | 0/10^{a} | 0/10^{a} | 0/5 |
| B | 3/5 | 3/5 | 1/5 | 1/5^{a} | 1/5^{a} | 0/5 | 1/5 | 1/5 |
| C | 12/12^{b} | 8/12^{b} | 4/12 | 9/10^{b} | 8/10^{b} | 5/10^{b} | 5/10^{b} | 1/5 |

^{a,b}indicate statistical significance of P < 0.05 for difference between means by Fisher's exact test ot proportions

TABLE 3
Salmonella isolation from tonsil

| | Day (Challenge = 0) | | | | | | | |
|---|---|---|---|---|---|---|---|---|
| Group | 2 | 5 | 7 | 9 | 11 | 15 | 21 | 23 |
| A | 6/10 | 1/7 | 2/7 | 1/7 | 0/5^{a} | 0/10^{a} | 0/10^{a} | 0/5 |
| B | NA | NA | NA | NA | NA | 2/5 | 2/5 | NA |
| C | 8/10 | 5/7 | 3/7 | 4/7 | 4/5^{b} | 7/10^{b} | 7/10^{b} | 3/5 |

^{a,b}indicate statistical significance of P < 0.05 for difference between means by Fisher's exact test of proportions

TABLE 4
Summary of Salmonella isolation from pig feces and tonsil

| | | Number of | Average Number of culture- | |
|---|---|---|---|---|
| Group | Parameter | Animals | positive days (std. Err) | |
| A | Pig feces | 10 | 3.3 | (1.96)^{a} |
| B | | 5 | 12.0 | (2.8) |
| C | | 10 | 17.5 | (1.96)^{b} |
| A | Tonsil | 5 | 4 | (1.4)^{a} |
| C | | 5 | 21 | (1.5)^{b} |

^{a,b}indicate statistical significance of P < 0.05 for difference between means by ANOVA

### EXAMPLE 4

As shown in FIG. 4, an in vitro spot test was conducted to evaluate potential bactericidal effects of Lactobacilli on S. typhimurium. In this test, 5 μl of overnight culture of an individual Lactobacillus strain were placed on the surface of MRS agar. After 18-hour incubation at 37° C., 5 ml of Lauria-Bertanelli top agar with 10⁸ cfu of S. typhimurium was poured over the surface of the Lactobacilli cultures on the MRS agar. Growth of S. typhimurium was inhibited by all five of the Lactobacillus strains tested as shown in FIG. 4.

The two strains of Lactobacilli (GS-1 and GS-2) being tested were resulted in reduced shedding of Salmonella at 2 days post-challenge (DPC). Administration of Lactobacillus spp. GS-1 and Lactobacillus spp. GS-2 to animals resulted in decreased antibody levels toward Salmonella compared to animals fed uninoculated milk cultures. Further, administration of Lactobacillus spp. GS-1 to animals resulted in decreased IgA concentration compared to animals fed uninoculated milk cultures. Administration of both strains of Lactobacillus resulted in decreased shedding of Salmonella in tonsil and fecal samples.

### EXAMPLE 5

A treatment regimen was designed as follows:

Group 1: Control-no Lactobacilli milk culture (n=5)

Group 2: Live Lactobacillus spp. GS-1 milk culture (n=5)

Group 3: Live Lactobacillus spp. GS-2 milk culture (n=5)

Group 4: Killed Lactobacillus spp. GS-1 milk culture (n=5)

Group 5: Killed Lactobacillus spp. GS-2 milk culture (n=5)

Pigs were acclimatized for approximately 12 days. Subsequently, each treatment group was fed respective milk cultures daily as described above for 28 days. Weights were compared before treatment and at the termination of the study on day 28.

The levels of S. typhimurium were reduced in tonsil and rectal samples as quickly as 48 hours after inoculation. Lactobacilli can thus reduce the rapid dissemination of Salmonella. This is advantageous considering the rapid dissemination of Salmonella in pigs that become contaminated during holding at the slaughterhouse.

### EXAMPLE 6

Germ-free (“GF”) pigs were obtained by surgically delivery and randomized into treatment groups and housed in sterile isolation chambers. Skin and rectal swabs were taken every 3-4 days to verify the germ-free isolators remain free of contaminates. Two days post surgery, two animals in groups 8 and 11 were given 5 to 10 mls of a probiotic cocktail via oral inoculation containing 1.6×10⁹ CFU/ml of Streptococcus alactolyticus isolated from the intestinal contents of a conventional pig; 2.2×10⁸ CFU/ml of Bacteriodes ovatus isolated from a Competitive Exclusion Culture provided by Dr. Robin Anderson from the University of Texas A&M; and 5.1×10⁸ CFU/ml of Lactobacillus paracasei paracasei strain GS-1 isolated from the intestinal wall of a conventional pig. Two days post surgery, two animals in groups 9 and 12 were given a Competitive Exclusion Culture (“CEC”) via oral inoculation. A CEC contains a mixture of unidentified microorganisms grown in mass fermentors. Two days post surgery, two animals in groups 7 and 10 were not inoculated with any probiotics and served a positive control. Four weeks after birth, the animals were inoculated intranasally with 5×10⁹ CFU/ml with a Salmonella enterica resistant to nalidixic acid and containing the gene for production of the green florescent protein. Three hours post infection, the animals were euthanized with pentobarbital sodium. The following tissue samples were aseptically collected for histopathology and reisolation of S. typhimurium: tonsil, mandibular lymph node, thymus, lung, liver, spleen, colon contents, ileocecal lymph node, cecum contents, and blood.

TABLE 5
Comparison of Salmonella isolation from pig
following administration of control, probiotic
cocktail, or undefined Competitive Exclusion Culture

| | | Group 8 | |
|---|---|---|---|
| | Group 7 and 10- | and 11- | |
| | no probiotic | probiotic | Group 9 and 12- |
| | cocktail or CEC | cocktail | CEC |
| Tonsil | 2/2 | 2/2 | 2/2 |
| Lung | 0/2 | 1/2 | 2/2 |
| Colon contents | 2/2 | 0/2 | 2/2 |
| Ileocecal Lymph | 0/2 | 0/2 | 2/2 |
| node | | | |
| Cecum contents | 2/2 | 0/2 | 2/2 |
| Percent RED | 60 | 30 | 100 |

As shown in Table 5, the probiotic cocktail reduced the rapid extraintestinal dissemination (“RED”) associated with Salmonella from 100% in the animals given the CEC to 30% in animals given the probiotic cocktail.

The above description and examples are only illustrative of embodiments which achieve the objects, features, and advantages of the present invention, and it is not intended that the present invention be limited thereto. Any modifications of the present invention which come within the spirit and scope of the following claims is considered part of the present invention.

What is claimed is:

1. A method of reducing the amount of Salmonella in a swine prior to shipment, comprising administering a composition containing lactic acid producing bacteria in an acceptable carrier to said swine at least about three weeks prior to shipment of said animal in an amount effective to reduce the amount of Salmonella in said animal, wherein said lactic acid producing bacteria consist essentially of Lactobacillus paracasei paracasei.

2. The method of claim 1, wherein said composition is administrated in an amount of about 1 to about 5000 mls containing lactic acid producing bacteria at a concentration of about 10² CFU/ml to about 10¹⁰ CFU/ml.

3. The method of claim 2, wherein said amount is about 500 to about 1000 mls.

4. The method of claim 1, wherein said composition is administered to said animal from about 48 hours to about three hours prior to shipment of said animal.

5. The method of claim 1, wherein said carrier comprises a material selected from the group consisting of a milk product, water, and animal feed.

6. The method of claim 5, wherein said milk product is yogurt.

7. The method of claim 1, wherein said composition is administered to said animal by oral or intranasal inoculation.

8. The method of claim 1, wherein said Salmonella is reduced by about 5 to 10 fold in the mesenteric lymph nodes of said animal.

9. The method of claim 1, wherein said Salmonella is reduced by about 1,000 to 10,000 fold in the cecum of said animal.

10. A composition for reducing the amount of Salmonella in swine consisting essentially of a Salmonella reducing effective amount of Lactobacillus paracasei paracasei in an acceptable carrier.

11. The composition of claim 10, wherein said effective amount of Lactobacillus paracasei paracasei is about 1 to about 5000 mls at a concentration of about 10² CFU/ml to about 10¹⁰ CFU/ml.

12. The composition of claim 11, wherein said amount is from at least about 500 to about 1000 mls.

13. The composition of claim 10, wherein said carrier is selected from group consisting of a milk product, water, and animal feed.

14. A method of reducing the amount of Salmonella in a livestock animal, comprising administering a Salmonella reducing effective amount of probiotic bacteria consisting essentially of lactic acid producing bacteria, acetic acid producing bacteria, and Bacteroides in an acceptable carrier.

15. The method of claim 14, wherein said lactic acid bacteria are selected from the group consisting of Lactobacillus, Lactococcus, Streptococcus, Bifidobacteria, and Proprionibateria, and said acetic acid producing bacteria are selected from the group consisting of Streptococcus, Peptostreptococcus, and Eubacterium.

16. The method of claim 14, wherein said lactic acid producing bacteria are administered to said animal in an amount of about 1 to about 5000 mls of said carrier having a concentration of about 10⁴ CFU/ml to about 10⁹ CFU/ml, said acetic acid producing acid bacteria are administered to said animal in an amount of about 1 to about 5000 mls of said carrier having a concentration of about 10⁴ CFU/ml to about 10⁹ CFU/ml, and said Bacteroides are administered to said animal in an amount of from about 1 to about 5000 mls of said carrier having a concentration of about 10⁴ CFU/ml to about 10⁹ CFU/ml.

17. The method of claim 16, wherein said amount of lactic acid producing bacteria is from at least about 5 to about 10 mls, said amount of acetic acid producing bacteria is from about 5 to about 10 mls, and said amount of Bacteroides is from about 5 to about 10 mls.

18. The method of claim 14, wherein said probiotic bacteria are administered to said animal at least daily from about three weeks to about three hours prior to transport of said animal.

19. The method of claim 14, wherein said probiotic bacteria are administered to said animal at least about 48 hours prior to transport of said animal.

20. The method of claim 14, wherein said lactic acid producing bacteria are administered to said animal by oral or intranasal inoculation.

21. A method of reducing the amount of Salmonella in a livestock animal, comprising administering a probiotic cocktail to an animal in an amount effective to reduce the amount of Salmonella in said animal, said probiotic cocktail consisting essentially of Bacteroides ovatus, Streptococcus alactolyticus, and Lactobacillus paracasei paracasei in an acceptable carrier.

22. The method of claim 21, wherein said Bacteroides ovatus are provided in an amount of from about 1 to about 5000 mls of said carrier having a concentration of at least about 10⁴ CFU/ml to about 10⁹ CFU/ml, said Streptococcus alactolyticus are provided in an amount of from about 1 to about 5000 mls of said carrier having a concentration of at least about 10⁴ CFU/ml to about 10⁹ CFU/ml, and said Lactobacillus paracasei paracasei are provided in an amount of from about 1 to about 5000 mls of said carrier having a concentration of at least about 10⁴ CFU/ml to about 10⁹ CFU/ml.

23. A composition for reducing the amount of Salmonella in a livestock animal consisting essentially of a Salmonella reducing effective amount of lactic acid producing bacteria, acetic acid producing bacteria, and Bacteroides in an acceptable carrier.

24. The composition of claim 23, wherein said lactic acid bacteria are Lactobacillus paracasei paracasei, said acetic acid producing bacteria are Streptococcus alactolyticus, and said Bacteroides are Bacteroides ovatus.

25. The composition of claim 23, wherein said lactic acid producing bacteria are provided in an amount of from about 1 to about 5000 mls of said carrier having a concentration of at least about 10⁴ CFU/ml to about 10⁹ CFU/ml, said acetic acid producing acid bacteria are provided in an amount of from about 1 to about 5000 mls of said carrier having a concentration of at least about 10⁴ CFU/ml to about 10⁹ CFU/ml, and said Bacteroides are provided in an amount of from about 1 to about 5000 mls of said carrier having a concentration of at least about 10⁴ CFU/ml to about 10⁹ CFU/ml.

26. The composition of claim 23, wherein said carrier is selected from group consisting of a milk product, water, and animal feed.

27. The composition of claim 26, wherein said milk product further comprises yogurt culture.

28. A kit for reducing the amount of Salmonella in a livestock animal comprising at least one container, wherein said at least one container contains a probiotic cocktail consisting essentially of lactic acid producing bacteria, acetic acid producing bacteria, and Bacteroides in an amount effective to reduce the amount of Salmonella in a livestock animal.

29. The kit of claim 28 wherein said probiotic bacteria are present in an amount of at least about 10⁴ CFU of Lactobacillus paracasei paracasei, at least about 10⁴ CFU of Streptococcus alactolyticus, and at least about 10⁴ CFU of Bacteroides ovatus.

30. The kit of claim 28 wherein said probiotic cocktail further comprises an acceptable carrier.

31. The kit of claim 30, wherein said carrier is selected from the group consisting of a milk product, water, and feed.

32. The kit of claim 31, wherein said milk product further comprises a yogurt culture.

33. A method of transporting a livestock animal comprising, administering a composition containing a lactic acid producing bacteria in an acceptable carrier to said animal prior to transport, wherein said lactic acid producing bacteria consist essentially of Lactobacillus paracasei paracasei, and transporting said animal.

34. A method of transporting a livestock animal comprising, administering a composition containing probiotic bacteria in an acceptable carrier to said animal prior to transport, wherein said probiotic bacteria consist essentially of Lactobacillus paracasei paracasei, Streptococcus alactolyticus, and Bacteroides ovatus, and transporting said animal.
